(19) United States
(12) Patent Application Publication
(10) Pub. No.: US 2014/0322735 A1
(43) Pub. Date: Oct. 30, 2014

(54) Method to Measure Endogenous Enzymatic Serum/Plasma Cholesterol Esterification by LCAT (Lecithin:Cholesterol Acyltransferase) Assay

(71) Applicant: VascularStrategies LLC, Plymouth Meeting, PA (US)

(72) Inventors: Steven Adelman, Doylestown, PA (US); Heidi Collins, Hatboro, PA (US)

(73) Assignee: VascularStrategies LLC, Plymouth Meeting, PA (US)

(21) Appl. No.: 14/262,720

(22) Filed: Apr. 26, 2014

Related U.S. Application Data
(60) Provisional application No. 61/816,358, filed on Apr. 26, 2013.

(51) Int. Cl. C12Q 1/60

(52) U.S. Cl. CPC C12Q 1/60
USPC 435/11

(57) ABSTRACT

The present invention provides a method for the assessment of cholesterol esterification through physiologically relevant pathways and incorporates the function of individual subject's endogenous HDL particles. This ex vivo approach avoids the use of an artificial substrate and provides for determination of LCAT activity that includes both the contribution of a subject's endogenous HDL function and endogenous LCAT protein, and is therefore a significant improvement to the biologic relevance.

### CROSS-REFERENCE TO RELATED APPLICATIONS

This application claims priority to U.S. Provisional Application No. 61/816,358 filed Apr. 26, 2013, which is hereby incorporated by reference.

### BACKGROUND OF THE INVENTION

Lecithin cholesterol acyltransferase (LCAT), a plasma enzyme secreted by hepatocytes, catalyzes the transfer of acyl group of fatty acids from the 2-sn position of lecithin to the 3-hydroxy group of cholesterol. The reaction takes place primarily on the surface of high-density lipoproteins (HDL). In human plasma, this reaction is the major source of cholesteryl esters (CE) and has a crucial role in the remodeling of plasma lipoproteins and in reverse cholesterol transport (RCT). RCT, a process for removal of cholesterol from cells of the vascular wall for transport to the blood, liver and ultimate fecal excretion, is expected to be of importance in the treatment of hyperlipidemia and atherosclerosis and of benefit for intervention in cardiovascular disease. HDL function is critical to this process, and an understanding of the individual steps involved in RCT, including the LCAT reaction, will be of significant help in our approaches to intervention.

HDL acquires unesterified cholesterol through a variety of transfer processes. This is accomplished largely through cellular cholesterol efflux mechanisms from cells such as macrophages or hepatocytes. Cholesterol efflux from cells to HDL is considered the initial step in RCT. Mechanisms for cellular cholesterol efflux include specific transporters such as ABCA1 and ABCG1, scavenger receptors such as SR-BI, as well as a non-specific process termed aqueous diffusion. Once unesterified cholesterol is transferred through these pathways, it is incorporated onto the lipoprotein particle where it is then available for the next phases of metabolic processing required for RCT, including LCAT mediated esterification.

During RCT, a portion of the unesterified cholesterol on HDL is esterified through the action of LCAT. Lecithin (or phosphatidyl choline) is also present on the HDL molecule, acquired through endogenous lipoprotein synthetic and transfer pathways. The LCAT/HDL cholesterol esterifying reaction has three phases: 1) activation of the phospholipid bilayer on the surface of HDL; 2) release of a fatty acyl molecule by hydrolysis of lecithin; and 3) transfer of the fatty acyl molecule to 3-hydroxy group of cholesterol. The precise composition and location of the component molecule(s) of HDL will influence the LCAT reaction, and therefore, will impact overall HDL function, lipid metabolism and RCT.

Although there is a definite potential for the clinical use of LCAT assessment, its use is not widespread and is currently limited to the diagnosis of LCAT deficiency syndromes such as LCAT deficiency and Fish Eye Disease. After almost 30 years of efforts to reach consensus on the best assessment of LCAT activity and/or the rate of cholesterol esterification in plasma, there is still considerable ambiguity and misunderstanding surrounding the assays used today. A number of reviews describe methods for LCAT mass and activity determination. It is of particular importance to find a method that reflects the physiological process and indicates the amount of CE produced.

Among current assays for determination of LCAT are: 1) ELISA methods for determination of LCAT protein concentration and 2) fluorescence-based assays for determination of LCAT activity using artificial substrates in non-natural systems. The ELISA protein determination is clearly of use, but lacks utility for assessment of overall LCAT activity and physiological utility. Using the ELISA protein assay, the presence of LCAT isoforms or mutants with differences in activity could be overlooked. Furthermore, the protein assay does not allow assessment of the influence of other plasma components, such as endogenous HDL, that have a direct impact on the rate of LCAT-mediated cholesterol esterification.

With respect to the current LCAT activity measurements, there is essentially a single in vitro approach that utilizes a non-physiological artificial substrate. In these assays, purified LCAT or a source of LCAT such as plasma, is incubated with a fluorescently labeled substrate added exogenously to the samples. LCAT then transfers an acyl chain from the artificial substrate's sn-2 position of phosphatidylcholine to cholesterol, mimicking the LCAT reaction. Although this reaction does appear to measure LCAT activity as it relates to LCAT protein content, it lacks the capacity to assess the influence of the endogenous components present in the subject sample on overall LCAT-mediated cholesterol esterification. The inability to assess the impact of the function of particular HDL or other lipoproteins present in the samples, limits the physiological relevance of this in vitro approach.

### SUMMARY OF THE INVENTION

In some embodiments, the present invention provides a method for the assessment of cholesterol esterification through physiologically relevant pathways and incorporates the function of individual subject's endogenous HDL particles. This ex vivo approach avoids the use of an artificial substrate and provides for determination of LCAT activity that includes both the contribution of a subject's endogenous HDL function and endogenous LCAT protein, and is therefore a significant improvement to the biologic relevance.

In some embodiments, the present invention provides for methods for measuring endogenous lecithin cholesterol acyltransferase (LCAT) activity in a subject's sample. In some embodiments, the present invention provides methods for determining the activity of LCAT in a sample. These methods detect and quantitate the cholesterol esterification activity of LCAT. In some embodiments, the methods enable the accurate determination of the amount of cholesteryl ester formed by measurement of labeled cholesteryl ester, thereby enabling detection of LCAT activity level in the sample.

### BRIEF DESCRIPTION OF THE FIGURES

FIG. 1 is a graph of global cholesterol efflux from J774 cells incubated with serum HDL.

FIG. 2 is a graph of ex vivo LCAT activity.

FIG. 3 is a graph of the correlation of ex vivo LCAT activity with LCAT mass in serums of subjects treated with human, recombinant LCAT (rhLCAT).

### DETAILED DESCRIPTION OF THE INVENTION

In some embodiments, the present invention provides for a method to measure endogenous lecithin cholesterol acyltransferase (LCAT) activity in a subject's sample. In some embodiments, the present invention utilizes a cholesterol tracer that is incorporated into the endogenous HDL in subject's samples using a biologically relevant method. In some embodiments, cells such as macrophages, similar to those existing in the arterial wall, are incubated with a cholesterol tracer. In some embodiments, following the cholesterol tracer's equilibration within the cells, the sample, such as serum HDL, is then added and the cholesterol tracer is transferred from cells to the sample's HDL through physiologically relevant pathways (ABCA1, SRB1, ABCG1 and aqueous diffusion). In some embodiments, using the subject's endogenous HDL particles with physiologically incorporated tracer cholesterol, the measurement of cholesterol esterification occurs via the endogenous LCAT present in the subject's sample. In some embodiment, this improved method allows for assessment of cholesterol esterification through physiologically relevant pathways and incorporates the function of individual subject's endogenous HDL particles into the reaction process. In some embodiments, this ex vivo approach avoids the use of an artificial or exogenous substrate and provides for determination of LCAT activity that includes both the contribution of a subject's or sample's endogenous HDL function and endogenous LCAT protein, and is therefore a significant improvement to the biologic relevance.

In some embodiments of the present invention, a method of determining the level of LCAT activity in a sample comprises incubating cells with a tracer, adding the sample to the cells, incubating the sample with the cells, and determining LCAT activity in the sample.

In some embodiments of the present invention, a method of determining the level of LCAT activity in a sample comprises incubating cells with a tracer, adding the sample to the cells, incubating the sample with the cells, and determining cholesterol esterification by LCAT activity in the sample.

In some embodiments of the present invention, a method of determining the level of LCAT activity in a sample comprises incubating cells with a tracer, adding the sample to the cells, incubating the sample with the cells, allowing for cholesterol to efflux to the media and determining cholesterol esterification by LCAT activity in the sample.

In some embodiments of the present invention, a method of determining the level of endogenous LCAT activity in a sample comprises incubating cells with a tracer, adding the sample to the cells, incubating the sample with the cells, and determining LCAT activity in the sample.

In some embodiments of the present invention, a method of determining the level of endogenous LCAT activity in a sample comprises incubating cells with a tracer, adding the sample to the cells, incubating the sample with the cells, and determining cholesterol esterification by LCAT activity in the sample.

In some embodiments of the present invention, a method of determining the level of endogenous LCAT activity in a sample comprises incubating cells with a tracer, adding the sample to the cells, incubating the sample with the cells, allowing for cholesterol to efflux to the media and determining cholesterol esterification by LCAT activity in the sample.

In some embodiments of the present invention, a method of determining the level of LCAT activity in a sample comprises incubating cells with a tracer, adding the sample to the cells, incubating the sample with the cells, and determining LCAT activity in the sample, wherein the method does not comprise an exogenous substrate.

In some embodiments of the present invention, a method of determining the level of LCAT activity in a sample comprises incubating cells with a tracer, adding the sample to the cells, incubating the sample with the cells, and determining LCAT activity in the sample, wherein the method does not comprise an exogenous substrate for LCAT.

In some embodiments of the present invention, a method of determining the level of LCAT activity in a sample comprises incubating cells with a cholesterol tracer, adding the sample to the cells, incubating the sample with the cells, and determining cholesterol esterification by LCAT activity in the sample.

In some embodiments of the present invention, a method of determining the level of LCAT activity in a sample comprises incubating cells with a cholesterol tracer, adding the sample to the cells, incubating the sample with the cells, allowing for cholesterol to efflux to the media and determining cholesterol esterification by LCAT activity in the sample.

In some embodiments of the present invention, a method of determining the level of LCAT activity in a sample comprises incubating cells with a cholesterol tracer for a period of time, adding the sample to the cells, incubating the sample with the cells for a period of time, and determining cholesterol esterification by LCAT activity in the sample.

In some embodiments of the present invention, a method of determining the level of LCAT activity in a sample comprises incubating cells with a tracer, adding the sample to the cells, incubating the sample with the cells, measuring the amount of cholesterol released, and determining the LCAT activity in the sample.

In some embodiments of the present invention, a method of determining the level of LCAT activity in a sample comprises incubating cells with a tracer, adding the sample to the cells, incubating the sample with the cells, measuring the amount of cholesterol released, and determining the LCAT activity in the sample.

In some embodiments of the present invention, a method of determining the level of LCAT activity in a sample comprises incubating cells with a tracer for a period of time, adding the sample to the cells, incubating the sample with the cells for a period of time, measuring the amount of cholesterol released, and determining the LCAT activity in the sample.

In some embodiments of the present invention, a method of determining the level of LCAT activity in a sample comprises incubating cells with a cholesterol tracer, adding the sample to the cells, incubating the sample with the cells, measuring the amount of cholesterol released, and determining the LCAT activity in the sample.

In some embodiments of the present invention, a method of determining the level of LCAT activity in a sample comprises incubating cells with a cholesterol tracer for a period of time, adding the sample to the cells, incubating the sample with the cells for a period of time, measuring the amount of cholesterol released, and determining the LCAT activity in the sample.

In some embodiments of the present invention, a method of determining the level of LCAT activity in a sample comprises incubating cells with radiolabeled cholesterol, adding the sample to the cells, measuring the amount of radiolabeled cholesterol released, and determining the LCAT activity in the sample.

In some embodiments of the present invention, a method of determining the level of LCAT activity in a sample comprises incubating cells with radiolabeled cholesterol, adding the sample to the cells, incubating the sample with the cells, and determining cholesterol esterification by LCAT activity in the sample.

In some embodiments of the present invention, a method of determining the level of LCAT activity in a sample comprises incubating cells with radiolabeled cholesterol, adding the sample to the cells, incubating the sample with the cells, allowing for cholesterol to efflux to the media and determining cholesterol esterification by LCAT activity in the sample.

In some embodiments of the present invention, a method of determining the level of LCAT activity in a sample comprises incubating cells with radiolabeled cholesterol for a period of time, adding the sample to the cells, incubating the sample with the cells for a period of time, measuring the amount of radiolabeled cholesterol released, and determining the LCAT activity in the sample.

In some embodiments of the present invention, a method of determining the level of LCAT activity in a sample comprises incubating cells with radiolabeled cholesterol, adding the sample to the cells, measuring the amount of radiolabeled cholesterol released, and determining the LCAT activity in the sample, wherein the sample is serum HDL.

In some embodiments, the method further comprises adding cyclic adenosine monophosphate (cAMP) to the cells after the incubation with the cholesterol tracer. In some embodiments, the method further comprises adding cyclic adenosine monophosphate (cAMP) to the cells after the completion of the incubation with the cholesterol tracer. In some embodiments, the method further comprises adding to and incubating cAMP with the cells after the incubation with the tracer. In some embodiments, the method further comprises adding cyclic adenosine monophosphate (cAMP) to the cells after the completion of the incubation with the tracer. In some embodiments, the method further comprises adding and incubating cAMP to the cells after the incubation with the cholesterol tracer.

In some embodiments, the method further comprises incubating cAMP with the cells concurrently with incubating the cells with the tracer. In some embodiments, the method further comprises incubating cAMP with the cells concurrently with incubating the cells with the cholesterol tracer.

In some embodiments, a method of determining LCAT activity in a sample comprises incubating cells with a tracer, incubating the cells with cAMP, adding the sample to the cells, and determining the LCAT activity in the sample. In some embodiments, a method of determining LCAT activity in a sample comprises incubating cells with a tracer, incubating the cells with cAMP, adding the sample to the cells, and determining cholesterol esterification by LCAT activity in the sample. In some embodiments, a method of determining LCAT activity in a sample comprises incubating cells with a tracer, incubating the cells with cAMP, adding the sample to the cells, removing an aliquot of the sample from the cells, extracting lipids from the aliquot and determining the LCAT activity in the sample. In some embodiments, a method of determining LCAT activity in a sample comprises incubating cells with a tracer and with cAMP, adding the sample to the cells, removing an aliquot of the sample from the cells, extracting lipids from the aliquot and determining the LCAT activity in the sample. In some embodiments, a method of determining LCAT activity in a sample comprises incubating cells with a tracer and with cAMP for a period of time, adding the sample to the cells for a period of time, removing an aliquot of the sample from the cells, extracting lipids from the aliquot and determining the LCAT activity in the sample. In some embodiments, a method of determining LCAT activity in a sample comprises incubating cells with a tracer and with cAMP, incubating the sample with the cells, removing an aliquot of the sample from the cells, extracting lipids from the aliquot and determining the LCAT activity in the sample. In some embodiments, a method of determining LCAT activity in a sample comprises incubating cells with a cholesterol tracer, incubating the cells with cAMP, adding the sample to the cells, removing an aliquot of the sample from the cells, extracting lipids from the aliquot and determining the LCAT activity in the sample.

In some embodiments of the present invention, the sample to be assayed is a biological fluid. In some embodiments, the biological fluid is human. In some embodiments, the biological fluid is from a non-human species. In some embodiments, the biological fluid is plasma. In other embodiments, the biological fluid is serum. In some embodiments, the biological fluid is blood. In some embodiments, the biological fluid is cerebral spinal fluid. In certain embodiments of the invention, the sample to be assayed is plasma or serum or blood. In some embodiments, the plasma or serum is human. In other embodiments, the plasma or serum is from a non-human species. In some embodiments of the invention, the sample is incubated with the cells from about 2 to about 8 hours.

In some embodiments, apolipoprotein B lipoproteins (apoB lipoproteins) are removed from the sample prior to incubation with the cells. The removal of apoB lipoproteins, such as low density lipoproteins (LDL) and very low density lipoproteins (VLDL), from the sample provides for the sample to retain all other endogenous components, including high density lipoproteins (HDL) and LCAT. Procedures for the removal of apoB lipoproteins from samples are well known in the art. In some embodiments of the present invention, the apoB lipoproteins are removed from the samples by precipitation. In some embodiments, the apoB lipoproteins are removed from the samples by precipitation with polyethylene glycol (PEG).

In some embodiments of the present invention, a method of determining the level of LCAT activity in a sample comprises incubating cells with a tracer, removing apoB lipoproteins from the sample, adding the sample to the cells, incubating the sample with the cells, and determining the LCAT activity in the sample.

In some embodiments of the present invention, a method of determining the level of LCAT activity in a sample comprises incubating cells with a tracer, removing apoB lipoproteins from the sample, adding the sample to the cells, incubating the sample with the cells, and determining cholesterol esterification by LCAT activity in the sample.

In some embodiments of the present invention, cells suitable for use may be a single population of cells of the same type, or may be a mixed population. In some embodiments, such cell types may be selected from adherent cells, for example mammalian cells. In some embodiments, cells may include, but are not limited to, stem cells, differentiated stem cells, primary cells, transformed cells and genetically engineered cells. In some embodiments, cells may include, but are not limited to, macrophages, fibroblasts, hepatocytes, adrenal cells, ovarian cells, testicular cells, and skin cells.

In some embodiments, tracers useful in the present invention include, but are not limited to, radiolabeled cholesterol, fatty acids or phospholipids; stable isotopically-labeled (heavy isotopes) cholesterol, fatty acids or phospholipids; and fluorescently-labeled cholesterol, fatty acids or phospholipids. In some embodiments, the fatty acids include, but are not limited to, linoleic acid, oleic acid, palmitic and stearic acid. In some embodiments, the phospholipids include, but are not limited to, phosphatidylcholine and phosphatidylserine, phosphatidylethanolamine, or phosphatidylinositol. In some embodiments of the present invention, the radiolabels include, but are not limited to, tritium (³H) and radiocarbon (¹⁴C). In some embodiments, the stable isotopic compounds include, but are not limited to, deuterated compounds such as ¹³C.

In some embodiments, the cells can be incubated with the tracer from about 2 to about 48 hours.

In some embodiments of the present method, the cholesteryl ester is measured by lipid extraction. In some embodiments, the lipid extraction is performed according to the Bligh and Dyer method. In some embodiments of the present invention, the lipid extraction is performed according to the Folch method. In some embodiments, the lipid extract is analyzed by thin layer chromatography (TLC). In some embodiments, the lipid extract is analyzed by high performance liquid chromatography (HPLC). In some embodiments, the lipid extract is analyzed by mass spectrometry. In some embodiments, the lipid extract is analyzed by infrared spectrometry. In some embodiments of the present invention, the LCAT activity is determined by calculating the proportion of the labeled cholesteryl ester to the total labeled cholesterol.

In some embodiments of the present invention, the methods are carried out using multi-well assay plates, e.g. 24-well, 96-well, as such the methods are well suited for use in high-throughput screening. Fluorescence may be measured by any suitable techniques, which are well known in the art. For example, an assay plate may be scanned in a fluorescence plate reader which is set at suitable excitation and emission wavelengths for the fluorescent tracer. The particular wavelengths may vary depending on the particular fluorescent tracer used.

In some embodiments, the method of the present invention are useful, for example, for the determination of LCAT levels in a subject's plasma or serum, for example to aid in the diagnosis of familial LCAT deficiency, of fish eye disease or other conditions of abnormal LCAT activity. In some embodiments, the methods of the present invention are useful for monitoring patients LCAT activity during therapy, following administration of drugs, including administration of LCAT. In some embodiments, the methods of the present invention are useful for monitoring LCAT activity in serum or plasma from non-human species.

### EXAMPLES

The invention now being generally described will be more readily understood by reference to the following examples, which are included merely for purposes of illustration of certain aspects and embodiments of the present invention and are not intended to limit the invention.

### Example 1

Serum HDL was prepared from individual serum samples by precipitation of apoB-containing lipoproteins using polyethylene glycol (PEG). Briefly, for each serum sample, 100 parts serum was mixed with 40 parts PEG (20%, v/v, in glycine buffer, pH 7.4). The mixture was incubated at room temperature for 20 minutes and then centrifuged at 10,000 rpm for 30 minutes at 4° C. The supernatant containing serum HDL was removed and stored at 4° C. overnight until used to prepare test samples.

Global cholesterol efflux to test samples was conducted. Briefly, J774 mouse macrophage cells per well were seeded on 24-well plates in growth medium. The next day, growth medium was replaced with labeling medium containing [³H]-cholesterol and ACAT inhibitor and incubated overnight. After the labeling period, cells were incubated with medium containing 0.2% (w/v) BSA and ACAT inhibitor for 16-18 Hrs. without (control) and with cAMP (upregulated). [³H]-cholesterol released to test samples after incubation with cAMP-treated J774 cells for 4 h was measured by liquid scintillation counting. Global cholesterol efflux is expressed as the radiolabel released as a percentage of [³H]-cholesterol within cells before addition of test samples. All efflux values were corrected by subtracting the small amount of radioactive cholesterol released from cells incubated with serum-free medium. In addition, ABCA1-dependent efflux is determined as the difference in efflux from cAMP-treated and untreated J774 cells for the assay controls only. Each efflux assay included two assay controls, 20 μg/ml ApoA-1 and 2% human serum pool. For ex vivo LCAT activity, lipids were extracted using the Bligh and Dyer method. The lipid extract was analyzed by thin layer chromatography and the proportion of [³H]-cholesterol released to serum HDL during the 4 h period that was esterified by serum LCAT was measured. Ex vivo LCAT activity is expressed as the [³H]-cholesterol ester as a percentage of the total [³H]-cholesterol. Each assay of ex vivo LCAT assay included two assay controls, 20 μg/mlApoA-1 and 2.8% human serum HDL.

### Precision

The first method validation parameter evaluated was assay precision, including both intra-assay repeatability and inter-assay precision of global cholesterol efflux and ex vivo LCAT activity. The intra-assay repeatability of six independent triplicate measurements was determined (Assay 1). The results for global cholesterol efflux from cAMP-treated J774 cells to serum HDL samples are shown in Table 1a. The acceptance criteria for intra-assay precision of global cholesterol efflux (≦20%) were met for all test samples ranged between 6-10%.

The results for ex vivo LCAT activity for serum HDL are shown in Table 1b. The acceptance criteria for intra-assay precision of ex vivo LCAT activity (≦20%) were met for all 6 serum HDL samples ranged between 3-10%.

The intermediate precision (inter-assay variability) was also determined for both global cholesterol efflux and ex vivo LCAT activity. The intermediate precision was determined both directly as measured and after normalization to the appropriate assay control. Results for the inter-assay variability of global cholesterol efflux from J774 cells to the serum HDL samples from four independent assays (Assays 1-4) are shown below in Tables 2a and 2b. The acceptance criteria for inter-assay precision as measured (≦30%) and after normalization (≦20%) were based on historical data for samples containing whole serum, lipoproteins or apolipoproteins. The acceptance criteria for intermediate precision of global cholesterol efflux across 4 assays as measured (Table 2a) and after normalization (Table 2b) were met for all samples.

Results for the inter-assay variability of ex vivo LCAT activity for serum HDL samples from four independent assays (Assays 1-4) are shown below in Tables 2c and 2d. The intermediate precision of ex vivo LCAT activity across assays 1-4 as measured (Table 2c) ranged from 6-22% CV, meeting acceptance criteria. After normalization (Table 2d), intermediate precision of ex vivo LCAT activity across assays 1-4 ranged from 6-13% CV, also meeting acceptance criteria.

### Limit of Quantitation (LOQ)

The second parameter of this method validation was the determination of the limit of quantitation (LOQ) for both global cholesterol efflux (Assay 5) and ex vivo LCAT activity (Assay 6). To determine the LOQ for global cholesterol efflux to serum HDL, 7 concentrations of serum HDL ranging from 0-1.4% were run for each of two lots of human serum. Results for assay 5 are shown in Table 3a. The calculated LOQ of global cholesterol efflux were determined to be 0.03% efflux for lot BRH510184 and 0.15% efflux for lot BRH510186. Given the results for both lots, the conservative value of 0.15% efflux for the LOQ for global cholesterol efflux to serum HDL was chosen. An LOQ of 0.15% efflux to serum HDL is consistent previously measured LOQ of cholesterol efflux to other types of acceptors such as whole serum and lipid-free apolipoproteins.

To determine the LOQ for ex vivo LCAT activity for serum HDL, 7 concentrations of lipoprotein-deficient serum (LPDS) ranging from 0-6% supplemented with 20 μg/ml apoA-I were analyzed in assay 6. Because it was determined that the relationship between ex vivo LCAT activity and LPDS concentration is only linear up to 4% LPDS, results for the 6 concentrations ranging from 0-4% were used to calculate the LOQ. Results for determination of LOQ in assay 6 are shown in Table 3b. Ex vivo LCAT activity results for all concentrations of LPDS above the LOQ were valid and the calculated LOQ of ex vivo LCAT activity was determined to be 0.40% ester.

### Linearity

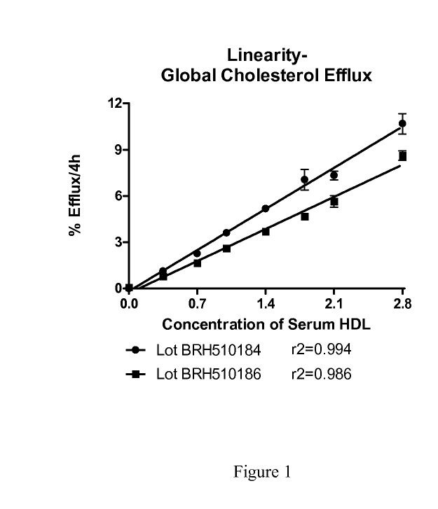

The linearity of global cholesterol efflux to serum HDL (Assay 5) and the linearity of ex vivo LCAT activity (Assay 6) were also assessed in this method validation. The acceptance criteria for linearity for both global cholesterol efflux and ex vivo LCAT activity is a linear correlation coefficient (r²) of ≧0.9 as determined by linear regression analysis. To evaluate the linearity of global cholesterol efflux to serum HDL, 8 concentrations of serum HDL ranging from 0-2.8% were run for each of two lots of human serum. Results of the linear regression analysis in assay 5 are shown in Table 4a and FIG. 1. Global cholesterol efflux was linear across the concentration range tested with linear correlation coefficients of r²=0.994 for lot BRH510184 and r²=0.987 lot BRH510186. Therefore, global cholesterol efflux to serum HDL results met the acceptance criteria for linearity.

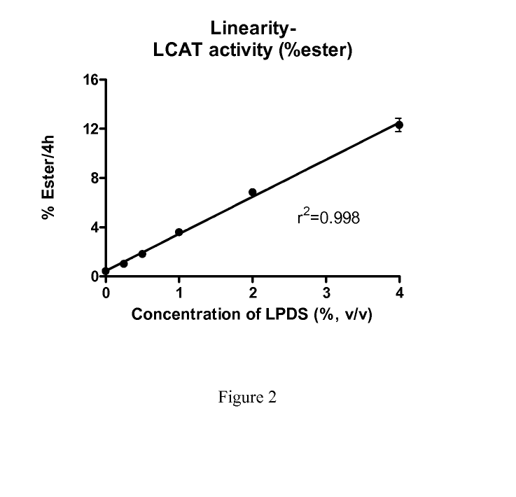

To evaluate the linearity for ex vivo LCAT activity, 8 concentrations of lipoprotein-deficient serum (LPDS) ranging from 0-8% supplemented with 20 μg/ml apoA-I were analyzed in assay 6. A visual examination of the graph shows that all data points are not linear. The data suggest that the percent ester begins to saturate at concentrations of LPDS greater than 4%, therefore the data points collected at 6% and 8% LPDS were excluded from the analysis. Linear Regression analysis of the relationship between LPDS concentration and the percent ester formed in 4 hours was determined for the remaining five concentrations of LPDS. Results for linearity in assay 6 are shown in Table 4b and FIG. 2. Ex vivo LCAT activity was linear across the 6 concentrations of LPDS tested with linear correlation coefficient of r²=0.998. Therefore ex vivo LCAT activity met the acceptance criteria for linearity in the presence of up to 4% serum.

### Specificity

In this method validation, specificity was also evaluated to demonstrate that global cholesterol efflux was specific to serum HDL. Specificity was evaluated by comparing the global cholesterol efflux to serum HDL to the global cholesterol efflux to blank media (Assays 1). For this assay of cholesterol efflux to be considered specific, the percentage of cholesterol efflux from cAMP-treated cells to serum HDL should be significantly higher than the percentage of cholesterol efflux to media blank in cAMP-treated cells as determined by two-tailed Student's t test with p<0.05. The results for global cholesterol efflux from cAMP-treated J774 cells to serum HDL samples and media blank are shown in Table 5a. The acceptance criteria for specificity of global cholesterol efflux were met for all test samples with p≦0.0001.

Specificity was also evaluated in the ex vivo LCAT activity assay to demonstrate that the % ester measured is due to the transfer activity of LCAT (Assay 1). For the assay of ex vivo LCAT activity to be considered specific, the % ester of serum HDL should be significantly higher than the % ester of serum HDL in the presence of LCAT inhibitor, 5,5′-dithiol-bis-nitrobenzoic acid (DTNB, Sigma-Aldrich), as determined by two-tailed Student's t test with p<0.05. The results of the ex vivo LCAT activity assay for serum HDL with and without DTNB are shown in Table 5b. The acceptance criteria for specificity of ex vivo LCAT activity were met for all 6 serum HDL samples (p≦0.0009). Overall the presence of the DTNB inhibited cholesterol esterification by 86-97% across all samples.

TABLE 1a
Intra-assay Repeatability of Global ³H-cholesterol Efflux from cAMP-treated
J774 cells to Serum HDL. Values reported are the average, standard deviation and % CV of 6
independent preparations of serum HDL for each lot of serum, each run in triplicate within a
single assay. Results are expressed as % efflux per 4 h. The acceptance criteria for intra-assay
repeatability of global cholesterol efflux is % CV ≦20%.

| | | | | | | | | % | % | % |
|---|---|---|---|---|---|---|---|---|---|---|
| | % | | | % | % | % | % | efflux | efflux | efflux |
| | Serum | % efflux | % efflux | efflux | efflux | efflux | efflux | +cAMP | +cAMP | +cAMP |
| Serum lot # | HDL | +cAMP 1 | +cAMP 2 | +cAMP 3 | +cAMP 4 | +cAMP 5 | +cAMP 6 | AVE | SD | % CV |
| BRH510182 | 2.8 | 9.46 | 8.55 | 7.31 | 7.97 | 7.53 | 7.82 | 8.11 | 0.79 | 10 |
| BRH510184 | 2.8 | 10.55 | 10.84 | 8.71 | 9.53 | 9.26 | 9.76 | 9.78 | 0.80 | 8 |
| BRH510186 | 2.8 | 7.14 | 6.75 | 7.92 | 7.83 | 7.64 | 7.71 | 7.50 | 0.46 | 6 |
| BRH510182 | 1.4 | 4.21 | 4.31 | 4.69 | 4.41 | 3.87 | 4.65 | 4.36 | 0.30 | 7 |
| BRH510184 | 1.4 | 4.91 | 4.83 | 5.35 | 5.16 | 5.92 | 4.82 | 5.17 | 0.42 | 8 |
| BRH510186 | 1.4 | 3.09 | 3.11 | 2.87 | 3.18 | 2.96 | 3.41 | 3.10 | 0.19 | 6 |

TABLE 1b
Intra-assay Repeatability of Ex Vivo LCAT Activity of Serum HDL. Values
reported are the average, standard deviation and % CV of 6 independent preparations of serum
HDL for each lot of serum, each run in triplicate within a single assay. Results are expressed as %
ester per 4 h. The acceptance criteria for intra-assay repeatability of ex vivo LCAT activity is % CV ≦20%.

| | | | | | | | | % ester | % ester | % ester |
|---|---|---|---|---|---|---|---|---|---|---|
| | % Serum | % ester | % ester | % ester | % ester | % ester | % ester | +cAMP | +cAMP | +cAMP |
| Serum lot # | HDL | +cAMP 1 | +cAMP 2 | +cAMP 3 | +cAMP 4 | +cAMP 5 | +cAMP 6 | AVE | SD | % CV |
| BRH510182 | 2.8 | 16.29 | 16.53 | 17.65 | 17.31 | 21.00 | 18.34 | 17.85 | 1.71 | 10 |
| BRH510184 | 2.8 | 12.86 | 13.11 | 12.37 | 12.85 | 12.61 | 13.61 | 12.90 | 0.43 | 3 |
| BRH510186 | 2.8 | 22.62 | 22.93 | 23.06 | 22.58 | 21.76 | 20.23 | 22.20 | 1.06 | 5 |
| BRH510182 | 1.4 | 11.90 | 12.12 | 11.72 | 12.84 | 11.56 | 11.60 | 11.96 | 0.48 | 4 |
| BRH510184 | 1.4 | 9.87 | 8.20 | 9.12 | 7.99 | 9.05 | 8.37 | 8.77 | 0.71 | 8 |
| BRH510186 | 1.4 | 16.94 | 15.81 | 16.53 | 16.41 | 17.52 | 15.05 | 16.38 | 0.86 | 5 |

TABLE 2a
Intermediate Precision of Global ³H-cholesterol Efflux from cAMP-
treated J774 cells to Serum HDL. Values reported are the average, standard deviation
and % CV of four independent assays of serum HDL for each lot of serum, each run in
triplicate per assay. Results are expressed as % efflux per 4 h as directly measured. The
acceptance criteria for intermediate precision of global cholesterol efflux as measured is
% CV ≦30%.

| | | % efflux | % efflux | % efflux | % efflux | % efflux | % efflux | % efflux |
|---|---|---|---|---|---|---|---|---|
| | % Serum | +cAMP | +cAMP | +cAMP | +cAMP | +cAMP | +cAMP | +cAMP |
| Serum lot # | HDL | Assay 1 | Assay 2 | Assay 3 | Assay4 | AVE | SD | % CV |
| BRH510182 | 2.8 | 9.46 | 10.20 | 7.34 | 8.59 | 8.90 | 1.23 | 14 |
| BRH510184 | 2.8 | 10.55 | 9.86 | 9.17 | 9.51 | 9.77 | 0.59 | 6 |
| BRH510186 | 2.8 | 7.14 | 7.73 | 5.02 | 7.37 | 6.82 | 1.22 | 18 |
| BRH510182 | 1.4 | 4.21 | 4.99 | 3.56 | 4.97 | 4.43 | 0.69 | 15 |
| BRH510184 | 1.4 | 4.91 | 5.33 | 4.69 | 4.82 | 4.94 | 0.28 | 6 |
| BRH510186 | 1.4 | 3.09 | 3.82 | 3.00 | 3.40 | 3.33 | 0.37 | 11 |

TABLE 2b
Intermediate Precision of Global ³H-cholesterol Efflux from cAMP-
treated J774 cells to Serum HDL after normalization. Values reported are the
average, standard deviation and % CV of four independent assays of serum HDL for each
lot of serum, each run in triplicate per assay. Results are expressed as % efflux per 4 h
after normalization to the human serum pool control. The acceptance criteria for
intermediate precision of global cholesterol efflux after normalization is % CV ≦20%.

| | | % efflux | % efflux | % efflux | % efflux | % efflux | % efflux | % efflux |
|---|---|---|---|---|---|---|---|---|
| | % Serum | +cAMP | +cAMP | +cAMP | +cAMP | +cAMP | +cAMP | +cAMP |
| Serum lot # | HDL | Assay 1 | Assay 2 | Assay 3 | Assay4 | AVE | SD | % CV |
| BRH510182 | 2.8 | 9.11 | 10.12 | 8.08 | 8.22 | 8.88 | 0.94 | 11 |
| BRH510184 | 2.8 | 10.16 | 9.78 | 10.09 | 9.10 | 9.78 | 0.48 | 5 |
| BRH510186 | 2.8 | 6.87 | 7.67 | 5.52 | 7.06 | 6.78 | 0.90 | 13 |
| BRH510182 | 1.4 | 4.05 | 4.95 | 3.92 | 4.76 | 4.42 | 0.51 | 12 |
| BRH510184 | 1.4 | 4.73 | 5.29 | 5.16 | 4.61 | 4.95 | 0.33 | 7 |
| BRH510186 | 1.4 | 2.98 | 3.79 | 3.30 | 3.25 | 3.33 | 0.34 | 10 |

TABLE 2c
Intermediate Precision of Ex Vivo LCAT Activity of Serum HDL. Values
reported are the average, standard deviation and % CV of four independent
assays of serum HDL for each lot of serum, each run in triplicate per
assay. Results are expressed as % ester per 4 h as directly measured. The
acceptance criteria for intermediate precision of ex vivo LCAT activity as
measured is % CV ≦30%.

| | | % ester | % ester | % ester | % ester | % ester | % ester | % ester |
|---|---|---|---|---|---|---|---|---|
| | % Serum | +cAMP | +cAMP | +cAMP | +cAMP | +cAMP | +cAMP | +cAMP |
| Serum lot # | HDL | Assay 1 | Assay 2 | Assay 3 | Assay4 | AVE | SD | % CV |
| BRH510182 | 2.8 | 16.29 | 16.43 | 21.59 | 15.59 | 17.48 | 2.77 | 16 |
| BRH510184 | 2.8 | 12.86 | 13.68 | 16.05 | 10.50 | 13.27 | 2.29 | 17 |
| BRH510186 | 2.8 | 22.62 | 22.44 | 22.80 | 19.92 | 21.95 | 1.36 | 6 |
| BRH510182 | 1.4 | 11.90 | 12.85 | 16.62 | 9.79 | 12.79 | 2.86 | 22 |
| BRH510184 | 1.4 | 9.87 | 10.81 | 13.53 | 9.24 | 10.86 | 1.89 | 17 |
| BRH510186 | 1.4 | 16.94 | 18.88 | 20.87 | 14.23 | 17.73 | 2.83 | 16 |

TABLE 2d
Intermediate Precision of Ex Vivo LCAT Activity of Serum HDL after
normalization. Values reported are the average, standard deviation and % CV of
four independent assays of serum HDL for each lot of serum, each run in
triplicate per assay. Results are expressed as % ester per 4 h after
normalization to the human serum pool HDL control. The acceptance criteria for
intermediate precision of ex vivo LCAT activity after normalization is
% CV ≦20%.

| | | % ester | % ester | % ester | % ester | % ester | % ester | % ester |
|---|---|---|---|---|---|---|---|---|
| | % Serum | +cAMP | +cAMP | +cAMP | +cAMP | +cAMP | +cAMP | +cAMP |
| Serum lot # | HDL | Assay 1 | Assay 2 | Assay 3 | Assay4 | AVE | SD | % CV |
| BRH510182 | 2.8 | 15.77 | 16.28 | 19.41 | 18.43 | 17.47 | 1.73 | 10 |
| BRH510184 | 2.8 | 12.45 | 13.56 | 14.43 | 12.42 | 13.21 | 0.97 | 7 |
| BRH510186 | 2.8 | 21.90 | 22.24 | 20.50 | 23.55 | 22.05 | 1.25 | 6 |
| BRH510182 | 1.4 | 11.52 | 12.73 | 14.95 | 11.58 | 12.69 | 1.60 | 13 |
| BRH510184 | 1.4 | 9.55 | 10.71 | 12.17 | 10.93 | 10.84 | 1.07 | 10 |
| BRH510186 | 1.4 | 16.40 | 18.71 | 18.77 | 16.83 | 17.67 | 1.24 | 7 |

TABLE 3a
LOQ of Global ³H-cholesterol Efflux from J774 cells to
serum HDL. Efflux values reported are the average and
standard deviation of triplicate measurements from a single
assay. LOQ is calculated as 10 times the SD of the blank
divided by the slope of the dose response.

| | |
|---|---|
| Lot BRH510184 | Lot BRH510186 |

| | % efflux | % efflux | % efflux | % efflux |
|---|---|---|---|---|
| % Serum | +cAMP | +cAMP | +cAMP | +cAMP |
| HDL | AVE | SD | AVE | SD |
| 0.00 | 0.01 | 0.01 | 0.07 | 0.04 |
| 0.05 | 0.13 | 0.04 | 0.18 | 0.12 |
| 0.10 | 0.44 | 0.04 | 0.24 | 0.05 |
| 0.20 | 0.53 | 0.08 | 0.45 | 0.04 |
| 0.35 | 1.12 | 0.11 | 0.79 | 0.12 |
| 0.70 | 2.27 | 0.09 | 1.88 | 0.40 |
| 1.4 | 5.18 | 0.33 | 3.68 | 0.19 |

| | | |
|---|---|---|
| Slope | 3.666 | 2.626 |
| LOQ | 0.03% efflux | 0.15% efflux |

TABLE 3b
LOQ of Ex Vivo LCAT activity. LCAT activity values reported are
the average and standard deviation of triplicate measurements
of % ester from a single assay. LOQ is calculated as 10 times
the SD of the blank divided by the slope of the dose response.

| % LPDS +20 | % ester | % efflux |
|---|---|---|
| μg/ml | +cAMP | +cAMP |
| ApoA-I | AVE | SD |
| 0.00 | 0.43 | 0.12 |
| 0.25 | 1.02 | 0.13 |
| 0.50 | 1.82 | 0.08 |
| 1.00 | 3.59 | 0.03 |
| 2.00 | 6.84 | 0.26 |
| 4.00 | 12.31 | 0.55 |

| | |
|---|---|
| Slope | 3.015 |
| LOQ | 0.40% ester |

TABLE 4a
FIG. 1. Linearity of Global ³H-cholesterol Efflux from J774
cells to serum HDL. Efflux values reported are the average
and standard deviation of triplicate measurements from a single
assay. The acceptance criteria for linearity of global cholesterol
efflux is a correlation coefficient of r² ≧ 0.9 as calculated
by linear regression analysis.

| | Lot BRH510184 | | Lot BRH510186 | |
|---|---|---|---|---|
| | % efflux | % efflux | % efflux | % efflux |
| % Serum | +cAMP | +cAMP | +cAMP | +cAMP |
| HDL | AVE | SD | AVE | SD |
| 0.00 | 0.01 | 0.01 | 0.07 | 0.04 |
| 0.35 | 1.12 | 0.11 | 0.79 | 0.12 |
| 0.7 | 2.27 | 0.09 | 1.66 | 0.16 |
| 1.0 | 3.61 | 0.21 | 2.61 | 0.04 |
| 1.4 | 5.18 | 0.33 | 3.68 | 0.19 |
| 1.8 | 7.06 | 1.15 | 4.68 | 0.21 |
| 2.1 | 7.33 | 0.50 | 5.64 | 0.65 |
| 2.8 | 10.68 | 1.13 | 8.62 | 0.52 |

| | | |
|---|---|---|
| Linearity r² | 0.994 | 0.986 |

TABLE 4b
FIG. 2. Linearity of Ex Vivo LCAT activity. LCAT activity
values reported are the average and standard deviation
of triplicate measurements of % ester from a single assay. The
acceptance criteria for linearity of ex vivo LCAT activity is
a correlation coefficient of r² ≧ 0.9 as calculated
by linear regression analysis.

| % LPDS +20 | % ester | % efflux |
|---|---|---|
| μg/ml | +cAMP | +cAMP |
| ApoA-I | AVE | SD |
| 0.00 | 0.43 | 0.12 |
| 0.25 | 1.02 | 0.13 |
| 0.50 | 1.82 | 0.08 |
| 1.00 | 3.59 | 0.03 |
| 2.00 | 6.84 | 0.26 |
| 4.00 | 12.31 | 0.55 |

| | |
|---|---|
| Linearity r² | 0.998 |

TABLE 5a
Specificity of Global Cholesterol Efflux to Serum HDL. Values reported
are the average and standard deviation of global cholesterol efflux to serum HDL for each
lot of serum, each run in triplicate within a single assay. Results are expressed as %
efflux per 4 h. Results for serum HDL samples are compared to media blank results using
Student's t test.

| | | % efflux | % efflux | % efflux | % efflux | % efflux | |
|---|---|---|---|---|---|---|---|
| | % Serum | +cAMP | +cAMP | +cAMP | +cAMP | +cAMP | Student's |
| Serum lot # | HDL | Well 1 | Well 2 | Well 3 | AVE | SD | t test |
| BRH510182 | 2.8 | 9.02 | 10.18 | 9.18 | 9.46 | 0.63 | 0.00001 |
| BRH510184 | 2.8 | 9.89 | 10.32 | 11.43 | 10.55 | 0.80 | 0.00002 |
| BRH510186 | 2.8 | 7.84 | 6.70 | 6.88 | 7.14 | 0.61 | 0.00004 |
| BRH510182 | 1.4 | 4.05 | 4.46 | 4.11 | 4.21 | 0.22 | 0.00001 |
| BRH510184 | 1.4 | 5.57 | 4.67 | 4.49 | 4.91 | 0.58 | 0.00013 |
| BRH510186 | 1.4 | 3.16 | 3.07 | 3.04 | 3.09 | 0.06 | 0.00000 |
| Media blank | — | 0.07 | 0.00 | 0.00 | 0.02 | 0.04 | — |

TABLE 5b
Specificity of Vivo LCAT Activity of Serum HDL. Values reported are the
average and standard deviation of ex vivo LCAT activity for serum HDL
for each lot of serum, each run in triplicate within a single assay.
Results are expressed as % ester per 4 h. Results for serum HDL samples
without the LCAT inhibitor DTNB are compared to results for serum HDL
samples with the LCAT inhibitor DTNB using Student's t test.

| | | % ester | % ester | % ester | % ester | % ester | |
|---|---|---|---|---|---|---|---|
| Serum ID −/+ | % Serum | +cAMP | +cAMP | +cAMP | +cAMP | +cAMP | Student's |
| DTNB | HDL | Well 1 | Well 2 | Well 3 | AVE | SD | t test |
| BRH510182 − | 2.8 | 15.31 | 16.68 | 16.88 | 16.29 | 0.86 | 0.00001 |
| BRH510182 + | 2.8 | 1.32 | 1.75 | 1.04 | 1.37 | 0.36 | |
| BRH510184 − | 2.8 | 12.01 | 13.48 | 13.09 | 12.86 | 0.76 | 0.00001 |
| BRH510184 + | 2.8 | 1.02 | 0.89 | 1.02 | 0.98 | 0.08 | |
| BRH510186 − | 2.8 | 21.34 | 23.59 | 22.94 | 22.62 | 1.16 | 0.00001 |
| BRH510186 + | 2.8 | 0.56 | 0.76 | 0.54 | 0.62 | 0.12 | |
| BRH510182 − | 1.4 | 11.98 | 12.07 | 11.66 | 11.90 | 0.21 | 0.00000 |
| BRH510182 + | 1.4 | 1.70 | 0.91 | 1.04 | 1.22 | 0.43 | |
| BRH510184 − | 1.4 | 9.76 | 9.62 | 10.23 | 9.87 | 0.32 | 0.00000 |
| BRH510184 + | 1.4 | 0.84 | 0.77 | 0.94 | 0.85 | 0.09 | |
| BRH510186 − | 1.4 | 14.32 | 20.36 | 16.14 | 16.94 | 3.10 | 0.00092 |
| BRH510186 + | 1.4 | 1.20 | 1.27 | 1.16 | 1.21 | 0.06 | |

### Conclusions

The results for Intra-assay repeatability of Global cholesterol efflux to all serum HDL test samples met the acceptance criteria. The results from 4 independent global cholesterol efflux assays do meet the acceptance criteria for Intermediate Precision both as measured and after normalization.

The results for Intra-assay repeatability of ex vivo LCAT activity for serum HDL in Assay 1 met the acceptance criteria for all 6 samples. The ex vivo LCAT activity results for all samples from Assays 1-4 did meet the acceptance criteria for Intermediate Precision both as measured and after normalization.

The LOQ for global cholesterol efflux was determined to be 0.15% efflux per 4 h. The LOQ for ex vivo LCAT activity was determined to be 0.40% ester per 4 h. Both global cholesterol efflux (r²=0.987-0.994) and ex vivo LCAT activity (r²=0.998) met the acceptance criteria for linearity. Global cholesterol efflux was determined to be specific for known cholesterol acceptors such as serum HDL (p≦0.0001 versus media blank). The cholesterol esterification measured in the ex vivo LCAT activity was determined to be specific for the action of LCAT because the presence of LCAT inhibitor decreased the % ester by 86-97% (p≦0.0009).

### Example 2

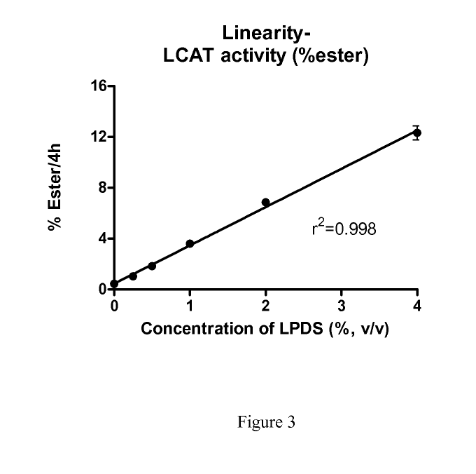

Human subjects were infused with 9 mg/kg ACP-501 (recombinant human LCAT (rhLCAT)) for 0, 1, 6, 12, 24, 48 and 72 hours. Serum samples were collected at each time point and LCAT mass and LCAT were determined. LCAT mass was determined by ELISA protein assay by AlphaCore Pharma. LCAT activity was determined as described in Example 1. FIG. 3 shows that ex vivo LCAT activity correlates with LCAT mass in serums of subjects treated with rhLCAT.

What is claimed is:

1. A method of determining the level of LCAT activity in a sample comprising incubating cells with a tracer, adding the sample to the cells, incubating the sample with the cells, and determining the LCAT activity in the sample.

2. The method of claim 1, wherein the sample is serum.

3. The method of claim 1, wherein the sample is plasma.

4. The method of claim 1, wherein the sample is blood.

5. The method of claim 1, wherein the sample is cerebrospinal fluid.

6. The method of claim 1, wherein the tracer is radiolabeled cholesterol, radiolabeled fatty acids, radiolabeled phospholipids, stable isotopically-labeled cholesterol, stable isotopically-labeled fatty acids, stable isotopically-labeled phospholipids, fluorescently-labeled cholesterol, fluorescently-labeled fatty acids or fluorescently-labeled phospholipids.

7. The method of claim 6, wherein the tracer is radiolabeled cholesterol.

8. The method of claim 6, wherein the tracer is fluorescently-labeled cholesterol.

9. The method of claim 1, wherein apolipoprotein B lipoproteins (apoB lipoproteins) are removed from the sample prior to incubating the sample with the cells.

10. The method of claim 9, wherein the apoB lipoproteins are removed from the sample by precipitation prior to incubating the sample with the cells.

11. The method of claim 1, wherein determining the LCAT activity in the sample comprises determining cholesterol esterification by LCAT activity in the sample.

12. The method of claim 1, wherein the method further comprises adding cyclic adenosine monophosphate (cAMP) to the cells after the incubation with the tracer.

13. A method of determining the level of LCAT activity in a sample comprising incubating cells with a tracer, adding the sample to the cells, incubating the sample with the cells, allowing for cholesterol to efflux to the cell media and determining cholesterol esterification by LCAT activity in the sample.
